(19) United States
(12) Patent Application Publication
(10) Pub. No.: US 2026/0047755 A1
(43) Pub. Date: Feb. 19, 2026

(54) DISPLAY APPARATUS AND CONTROL METHOD THEREFOR

(71) Applicants: SAMSUNG ELECTRONICS CO., LTD., Suwon-si (KR); SAMSUNG LIFE PUBLIC WELFARE FOUNDATION, Seoul (KR)

(72) Inventors: Jaesung PARK, Suwon-si (KR); Yunghoon JEONG, Suwon-si (KR); Kyung Ah PARK, Suwon-si (KR); Sei Yeul OH, Suwon-si (KR); Don II HAM, Suwon-si (KR); Youngsu MOON, Suwon-si (KR)

(73) Assignees: SAMSUNG ELECTRONICS CO., LTD., Suwon-si (KR); SAMSUNG LIFE PUBLIC WELFARE FOUNDATION, Seoul (KR)

(21) Appl. No.: 19/368,745

(22) Filed: Oct. 24, 2025

Related U.S. Application Data
(63) Continuation of application No. PCT/KR2024/003504, filed on Mar. 20, 2024.

(30) Foreign Application Priority Data
Apr. 25, 2023 (KR) 10-2023-0054296

(51) Int. Cl. A61B 3/032; A61B 3/02; G06T 7/00; G06V 10/82

(52) U.S. Cl. CPC A61B 3/032; A61B 3/022; G06T 7/0002; G06V 10/82; G06T 2207/30168

(57) ABSTRACT

A display apparatus includes: a display; a user interface; a memory storing a contrast sensitivity test image and a contour sensitivity test image for measuring a visual acuity level of a user; and at least one processor configured to: through the user interface, receive a user input for identifying visual acuity information of the user, in a state in which the contrast sensitivity test image and the contour sensitivity test image are displayed on the display, based on the information stored in the memory, identify a plurality of sample images having different image qualities and corresponding to a plurality of image types based on the identified visual acuity information, in a state in which the visual acuity information of the user is identified based on the user input, identify image quality information corresponding to each of the plurality of image types.

### CROSS-REFERENCE TO RELATED APPLICATION(S)

This application is a continuation of International Application No. PCT/KR2024/003504, filed on Mar. 20, 2024, in the Korean Intellectual Property Receiving Office, which is based on and claims priority to Korean Patent Application No. 10-2023-0054296, filed on Apr. 25, 2023, in the Korean Intellectual Property Office, the disclosures of which are incorporated by reference herein in their entireties.

### BACKGROUND

### 1. Field

The present disclosure relates to a display apparatus and a control method therefor, and more particularly, to a method of performing image quality processing to match a user characteristic by using a contrast sensitivity test and a contour sensitivity test.

### 2. Description of Related Art

A visual acuity level of a visually impaired person may be diagnosed based on a vision test chart, and a general vision test may include black letters on a white background. However, in some cases, brightness, color, and saturation may serve major functions in recognizing an object. Among these factors, for the visually impaired person, distinction of light and dark by the presence or absence of light and distinction of colors may serve important functions in watching an image. Accordingly, a method has been developed to provide an image in which image quality processing is performed to prevent the visually impaired person from having any difficulty in watching the image.

Ability related to whether the visually impaired person may clearly identify an object that does not stand out from a background is referred to as contrast sensitivity ability. A contrast sensitivity test is mainly used as a method for measuring the contrast sensitivity ability. However, an exact visual acuity level of the visually impaired person may not be easily identified based solely on the contrast sensitivity test.

Accordingly, a method is needed for detecting a contour emphasis level appropriate for an individual visual impairment level by including the contrast sensitivity ability, and for using the detected data to provide a visibility enhancement image quality service that matches characteristics of each individual visually impaired person and that is optimized for each individual visually impaired person.

### SUMMARY

Aspects will be set forth in part in the description which follows and, in part, will be apparent from the description, or may be learned by practice of the presented embodiments.

According to an aspect of the disclosure, a display apparatus may include: a display; a user interface; a memory storing a contrast sensitivity test image and a contour sensitivity test image for measuring a visual acuity level of a user; and at least one processor configured to: through the user interface, receive a user input for identifying visual acuity information of the user, in a state in which the contrast sensitivity test image and the contour sensitivity test image are displayed on the display, based on the information stored in the memory, identify a plurality of sample images having different image qualities and corresponding to a plurality of image types based on the identified visual acuity information, in a state in which the visual acuity information of the user is identified based on the user input, identify image quality information corresponding to each of the plurality of image types based on a second user input for selecting at least one of the plurality of sample images, in a state in which the plurality of sample images are displayed on the display, and display an image on the display based on the identified image quality information.

The at least one processor may be further configured to: identify first visual acuity information corresponding to a contrast sensitivity of the user based on the user input received through the user interface, in a state in which the contrast sensitivity test image is displayed on the display, provide, on the display, a contour sensitivity test image corresponding to the first visual acuity information, in a state in which the contour sensitivity test image is identified based on the identified first visual acuity information, and identify second visual acuity information corresponding to a contour sensitivity of the user based on the user input received through the user interface, in a state in which the contour sensitivity test image corresponding to the first visual acuity information is displayed.

The memory may be further storing a plurality of contrast sensitivity test images respectively corresponding to a plurality of contrast sensitivity levels, and a plurality of contour sensitivity test images having different contour sensitivities that respectively correspond to the plurality of contrast sensitivity test images, where the at least one processor is configured to: identify a contrast sensitivity level corresponding to a first contrast sensitivity test image as the first visual acuity information based on the information stored in the memory, in a state in which a contrast sensitivity of the first contrast sensitivity test image is unidentifiable from the plurality of contrast sensitivity test images based on the user input received through the user interface, and identify the second visual acuity information based on the user input received through the user interface, in a state in which at least one contour sensitivity test image corresponding to the first visual acuity information is displayed based on the information stored in the memory.

The plurality of image types may include a face type, a building type, a natural scene type, and a graphic type, where the memory is further storing the plurality of sample images having different image qualities and corresponding to the plurality of image types based on the visual acuity information of the user, and where the at least one processor is further configured to identify a plurality of sample images having different image qualities and corresponding to the face type, the building type, the natural scene type, and the graphic type based on the information stored in the memory, in a state in which the visual acuity information of the user is identified based on the user input.

The apparatus may further include a communication interface, where the memory is further storing a function corresponding to a correlation between the visual acuity information of the user and an image quality of an image, and where the at least one processor is further configured to: identify the plurality of sample images having different image qualities and corresponding to the plurality of image types, and the image quality information corresponding to each of the plurality of image types, by inputting, into the function, user context information and the identified visual acuity information of the user received through the communication interface, and identify image quality information corresponding to at least one selected sample image as the image quality information, in a state in which the second user input for selecting at least one of the plurality of sample images is received through the user interface.

The at least one processor may be further configured to identify image quality information corresponding to one of the at least one selected sample image as the image quality information, in a state in which the user input for selecting one of the sample images respectively corresponding the plurality of image types is received through the user interface.

The memory may be further storing a neural network model trained to classify types of the image displayed on the display apparatus, where the at least one processor is further configured to: identify a type of the displayed image by inputting information about an image provided on the display into the trained neural network model, and provide an image having an updated image quality on the display based on the image quality information corresponding to the identified image type.

The at least one processor may be further configured to provide, on the display, a second user interface for selecting at least one of the plurality of sample images.

The at least one processor may be further configured to: provide, on the display, a third user interface including a plurality of sample images corresponding to a first type among the plurality of image types, identify image quality information corresponding to a first image as image quality information corresponding to the first type, based on a user input received through the user interface for selecting the first image from the plurality of sample images corresponding to the first type, provide, on the display, a fourth user interface including a plurality of sample images corresponding to a second type among the plurality of image types, and identify image quality information corresponding to a second image as image quality information corresponding to the second type, based on a user input received through the second user interface for selecting the second image from the plurality of sample images corresponding to the second type.

According to an aspect of the disclosure, a control method for a display apparatus may include: receiving, through a user interface, a user input for identifying visual acuity information of a user, in a state in which a contrast sensitivity test image and a contour sensitivity test image for measuring a visual acuity level of the user are displayed on a display; identifying a plurality of sample images having different image qualities and corresponding to a plurality of image types based on the identified visual acuity information, in a state in which the visual acuity information of the user is identified based on the user input; identifying image quality information corresponding to each of the plurality of image types based on a second user input for selecting at least one of the plurality of sample images, in a state in which the plurality of sample images are displayed on the display; and displaying an image on the display based on the identified image quality information.

The receiving may include: identifying first visual acuity information corresponding to a contrast sensitivity of the user based on the user input received through the user interface, in a state in which the contrast sensitivity test image is displayed on the display, providing, on the display, a contour sensitivity test image corresponding to the first visual acuity information, in a state in which the contour sensitivity test image is identified based on the identified first visual acuity information, and identifying second visual acuity information corresponding to a contour sensitivity of the user based on the user input received through the user interface, in a state in which the contour sensitivity test image corresponding to the first visual acuity information is displayed.

The method may further include displaying, on the display, a plurality of contrast sensitivity test images respectively corresponding to a plurality of contrast sensitivity levels, and a plurality of contour sensitivity test images having different contour sensitivities that respectively correspond to the plurality of contrast sensitivity test images, based on the information stored in a memory, where, the identifying the first visual acuity information includes: identifying a contrast sensitivity level corresponding to a first contrast sensitivity test image as the first visual acuity information based on the information stored in the memory, in a state in which a contrast sensitivity of the first contrast sensitivity test image is unidentifiable from the plurality of contrast sensitivity test images based on the user input received through the user interface, and where the identifying the second visual acuity information includes: identifying the second visual acuity information corresponding to the contour sensitivity of the user based on the user input received through the user interface in a state in which at least one contour sensitivity test image corresponding to the first visual acuity information is displayed based on the information stored in the memory.

The plurality of image types may include a face type, a building type, a natural scene type, and a graphic type, where the identifying the plurality of sample images having the different image qualities include: identifying the plurality of sample images having different image qualities and corresponding to the face type, the building type, the natural scene type, and the graphic type based on the plurality of sample images having different image qualities that respectively correspond to the plurality of image types mapped to the visual acuity information of the user, stored in a memory, in a state in which the visual acuity information of the user is identified based on the received user input.

The method may further include identifying the plurality of sample images having different image qualities and corresponding to the plurality of image types, and the image quality information corresponding to each of the plurality of sample images having different image qualities, by inputting, into a function corresponding to a correlation between the visual acuity information of the user and an image quality of an image, user context information and the identified visual acuity information of the user, received through a communication interface, where the identifying the image quality information corresponding to each of the plurality of image types includes: identifying image quality information corresponding to at least one selected sample image as the image quality information corresponding, in a state in which the second user input for selecting at least one of the plurality of sample images is received through the user interface.

According to an aspect of the disclosure, provided a non-transitory computer-readable recording medium storing computer instructions that cause a display apparatus to perform an operation if executed by a processor of the display apparatus, and the operation may include: receiving, through a user interface, a user input for identifying visual acuity information of a user, in a state in which a contrast sensitivity test image and a contour sensitivity test image for measuring a visual acuity level of the user are displayed on a display, identifying a plurality of sample images having different image qualities and corresponding to a plurality of image types based on the identified visual acuity information, in a state in which the visual acuity information of the user is identified based on the user input, identifying image quality information corresponding to each of the plurality of image types based on a second user input for selecting at least one of the plurality of sample images, in a state in which the plurality of sample images are displayed on the display, and displaying an image on the display based on the identified image quality information.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a diagram for schematically describing a control method for a display apparatus according to an embodiment;

FIG. 2 is a block diagram showing a configuration of a display apparatus according to an embodiment;

FIG. 3 is a flowchart for describing the control method for a display apparatus according to an embodiment;

FIG. 4A and FIG. 4B are diagrams for describing a method for identifying visual acuity information according to an embodiment;

FIG. 5 is a diagram for describing a method for identifying the visual acuity information according to an embodiment;

FIG. 6 is a diagram for describing a method for identifying a plurality of sample images and image quality information according to an embodiment;

FIG. 7 is a diagram for describing a method for providing an updated image by using a neural network model according to an embodiment;

FIG. 8 is a diagram for describing a method for receiving a user input for selecting at least one of the plurality of sample images according to an embodiment;

FIG. 9A, FIG. 9B, FIG. 9C, and FIG. 9D are diagrams for describing a method for providing a user interface (UI) according to an embodiment; and

FIG. 10 is a block diagram showing a specific configuration of a display apparatus according to an embodiment.

### DETAILED DESCRIPTION

Hereinafter, the embodiments of the present disclosure are described in detail with reference to the accompanying drawings.

Terms used in the specification are briefly described, and the present disclosure is then described in detail.

General terms that are currently widely used are selected as terms used in embodiments of the present disclosure in consideration of their functions in the present disclosure, and may be changed based on the intention of those skilled in the art or a judicial precedent, the emergence of a new technique, or the like. In addition, in a specific case, terms arbitrarily chosen by an applicant may exist. In this case, the meanings of such terms are mentioned in detail in corresponding descriptions of the present disclosure. Therefore, the terms used in the present disclosure need to be defined on the basis of the meanings of the terms and the contents throughout the present disclosure rather than simple names of the terms.

In the present disclosure, an expression “have”, “may have”, “include”, “may include”, “comprise”, or “may comprise” or the like, indicates existence of a corresponding feature (for example, a numerical value, a function, an operation or a component such as a part), and does not exclude existence of an additional feature.

An expression, “at least one of A or/and B” may indicate either “A or B”, or “both of A and B.”

Expressions “first”, “second” and the like, used in the present disclosure may indicate various components regardless of the sequence or importance of the components. The expression is used only to distinguish one component from another component, and does not limit the corresponding component.

If any component (for example, a first component) is mentioned to be “(operatively or communicatively) coupled with/to” or “connected to” another component (for example, a second component), it should be understood that the any component is directly coupled to another component or may be coupled to another component through yet another component (for example, a third component).

A term of a singular number may include its plural number unless explicitly indicated otherwise in the context.

In the present disclosure, a “module” or a “˜er/˜or” may perform at least one function or operation, and be implemented by hardware, software, or a combination of hardware and software. In addition, a plurality of “modules” or a plurality of “˜ers/˜ors” may be integrated in at least one module and be implemented by the processor except for a “module” or a “˜er/or” that needs to be implemented by a specific hardware.

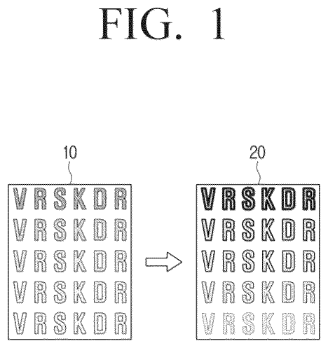

FIG. 1 is a diagram for schematically describing a control method for a display apparatus according to an embodiment.

A contrast sensitivity test may mainly be used as a method for measuring a contrast sensitivity ability of a user. The contrast sensitivity test refers to a test to measure a visual ability of the user by using a spatial frequency and changes in brightness and contrast of an object in an image. Meanwhile, a contour sensitivity test refers to a test to detect a contour emphasis level appropriate for a visual acuity level of the user. A visually impaired person may have a relatively low contrast sensitivity level in an image compared to a general public, and the visually impaired person may have an enhanced visibility in case of watching an image having an emphasized contour. That is, if the user has difficulty in recognizing an image at a specific contrast sensitivity level, a display apparatus 100 may apply a contour emphasis function to the image.

Referring to FIG. 1, according to an embodiment, the display apparatus 100 may identify visual acuity information of the user by displaying a contrast sensitivity test image 10 and a contour sensitivity test image 20.

According to an embodiment, the display apparatus 100 may identify a plurality of sample images having different image qualities based on the identified visual acuity information, and provide the user with the plurality of sample images. Here, the visual acuity information may include information about a contrast sensitivity level of the user and a contour sensitivity level of the user (or a contour emphasis level preferred by the user). Meanwhile, the plurality of sample images are sample images each reflecting the visual acuity level of the user.

According to an embodiment, the display apparatus 100 may identify an image quality level preferred by the user based on a selected sample image if one of the plurality of sample images is selected. For example, the display apparatus 100 may identify an image quality level corresponding to the selected sample image from the plurality of sample images having different image qualities. The display apparatus 100 may provide the user with an image corresponding to the identified image quality level.

Hereinafter, the description describes various embodiments in which the visual acuity information of the user is obtained through the contrast sensitivity test and the contour sensitivity test, the user is provided with the sample image used for identifying an image quality preferred by the user based on the obtained visual acuity information, and the image quality information preferred by the user is identified based on the obtained sample image. In addition, the description describes various embodiments in which the user is provided with an image reflecting the visual acuity level of the user and a user preference by providing an image reflecting the image quality preferred by the user.

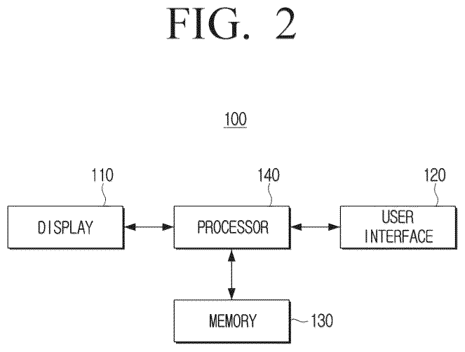

FIG. 2 is a block diagram showing a configuration of a display apparatus according to an embodiment.

Referring to FIG. 2, the display apparatus 100 may include a display 110, a user interface 120, a memory 130, and at least one processor 140.

The display apparatus 100 may be implemented as any of various apparatuses including a display and capable of playing image content, such as a smart television (TV), a tablet, a monitor, a smartphone, a desktop computer, and a laptop computer according to an embodiment. The display apparatus 100 according to an embodiment of the present disclosure is not limited to the above-described apparatuses, and the display apparatus 100 may be implemented as the display apparatus 100 having two or more functions of the above-described apparatuses.

Meanwhile, the display apparatus 100 may communicate with an external apparatus and an external server in various ways. According to an embodiment, a communication module for communicating with the external apparatus and the external server may be implemented identically. For example, the display apparatus 100 may communicate with the external apparatus by using a Bluetooth module, and also communicate with the external server by using the Bluetooth module.

The display 110 may be implemented as a display including a self-luminous element, or a display including a non-luminous element and a backlight. For example, the display 110 may be implemented as any of various forms of displays such as a liquid crystal display (LCD), an organic light-emitting diode (OLED) display, a light-emitting diode (LED) display, a micro LED, a Mini LED, a plasma display panel (PDP), a quantum dot (QD) display, and a quantum dot light-emitting diode (QLED) display. The display 110 may also include a driving circuit, a backlight unit, and the like, which may be implemented in a form such as an a-si thin film transistor (TFT), a low temperature poly silicon (LTPS) TFT, or an organic TFT (OTFT).

Meanwhile, the display 110 may be implemented as a touchscreen combined with a touch sensor, a flexible display, a rollable display, a three-dimensional (3D) display, a display in which a plurality of display modules are physically connected with each other, or the like. The processor 140 may control the display 110 to output an output image obtained according to the various embodiments described above. Here, the output image may be a high-resolution image of 4K, 8K, or higher.

The user interface 120 is a component for enabling the display apparatus 100 to perform interaction with the user. For example, the user interface 120 may include at least one of a touch sensor, a motion sensor, a button, a jog dial, a switch, a microphone, or a speaker, and is not limited thereto.

The memory 130 may store data required for the various embodiments. The memory 130 may be implemented as a memory embedded in the display apparatus 100 or as a memory detachable from the display apparatus 100 depending on a purpose of the data storage. For example, data for operating the display apparatus 100 may be stored in the memory embedded in the display apparatus 100, and data for expanding the display apparatus 100 may be stored in the memory detachable from the display apparatus 100.

Meanwhile, the memory embedded in the display apparatus 100 may be implemented as at least one of a volatile memory (e.g., a dynamic RAM (DRAM), a static RAM (SRAM), or a synchronous dynamic RAM (SDRAM)) or a non-volatile memory (e.g., a one time programmable ROM (OTPROM)), a programmable ROM (PROM), an erasable and programmable ROM (EPROM), an electrically erasable and programmable ROM (EEPROM), a mask ROM, a flash ROM, a flash memory (e.g., a NAND flash or a NOR flash), a hard drive, or a solid state drive (SSD)). In addition, the memory detachable from the display apparatus 100 may be implemented as a memory card (e.g., a compact flash (CF), a secure digital (SD), a micro secure digital (Micro-SD), a mini secure digital (Mini-SD), an extreme digital (xD), or a multi-media card (MMC)) or an external memory capable of being connected to a universal serial bus (USB) port (e.g., a USB memory).

According to an embodiment, the memory 130 may store an image for performing the contrast sensitivity test and an image for performing the contour sensitivity test to measure the visual acuity level of the user. Meanwhile, the contrast sensitivity test image may include at least one of a static contrast sensitivity image and a dynamic contrast sensitivity image. Here, the static contrast sensitivity image refers to an image that includes a stationary object, and the dynamic contrast sensitivity image refers to an image that includes a moving object.

Meanwhile, for example, the image for performing the contrast sensitivity test may be a left image in FIG. 1, and is not limited thereto. For example, the memory 130 may store the contrast sensitivity test image respectively corresponding to the plurality of contrast sensitivity levels. For example, the memory 130 may also store the image for performing the contour sensitivity test, and pre-store an image corresponding to each of the plurality of contour sensitivity levels. The user may sequentially watch a plurality of step-by-step images to identify a limit contrast sensitivity of the user and optimal contour sensitivity, and identify the contrast sensitivity level and contour sensitivity level of the user based on an identification ability of the user. This configuration is described below.

At least one processor 140 (hereinafter, the processor) may be electrically connected to the display 110, the user interface 120, and the memory 130, and control overall operations of the display apparatus 100. The processor 140 may include one or more processors. In detail, the processor 140 may execute at least one instruction stored in the memory 130, thereby perform the operations of the display apparatus 100 according to the various embodiments of the present disclosure.

The processor 140 according to an embodiment may be implemented as a digital signal processor (DSP), a microprocessor, a graphic processing unit (GPU), an artificial intelligence (AI) processor, a neural processing unit (NPU), a timing controller (TCON), each of which processes a digital signal. However, the processor 140 is not limited thereto, and may include at least one of a central processing unit (CPU), a micro controller unit (MCU), a micro processing unit (MPU), a controller, an application processor (AP), or a communication processor (CP), an advanced RISC machine (ARM) processor, or may be defined by a relevant term. In addition, the processor 140 may be implemented as a system-on-chip (SoC), a large scale integration (LSI) that has a processing algorithm embedded therein, or may be implemented in the form of an application specific integrated circuit (ASIC) or a field programmable gate array (FPGA).

The processor 140 according to an embodiment may display the contrast sensitivity test image and the contour sensitivity test image on the display 110. For example, the memory 130 may pre-store the contrast sensitivity test image and the contour sensitivity test image for measuring the visual acuity level of the user, and the processor 140 may display the contrast sensitivity test image and the contour sensitivity test image on the display 110 based on the information stored in the memory 130.

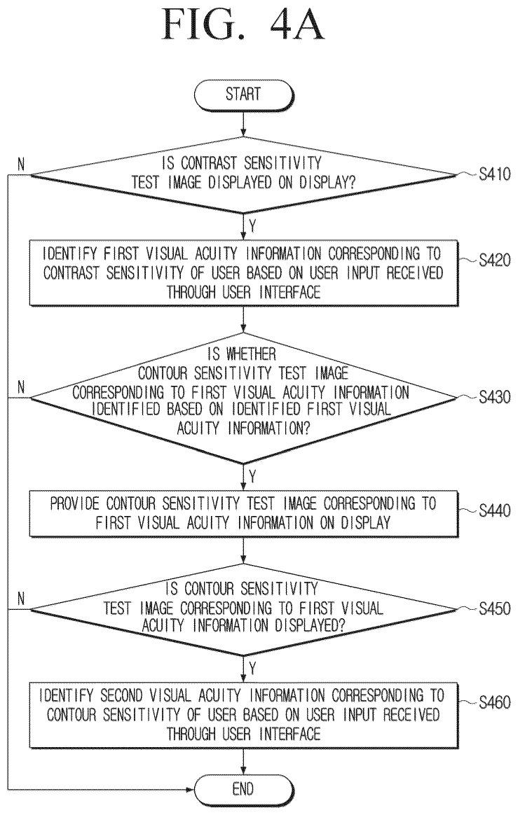

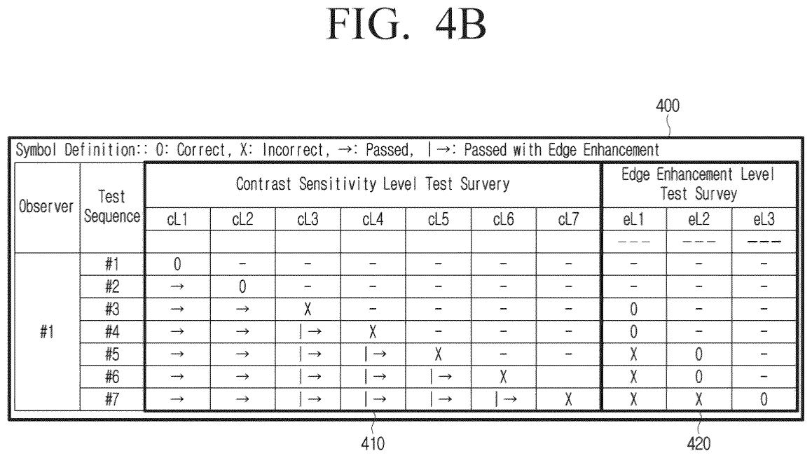

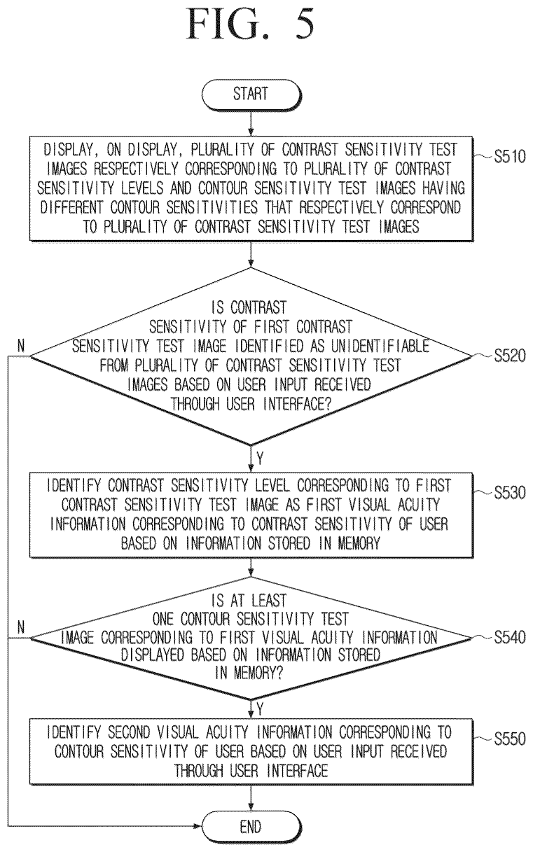

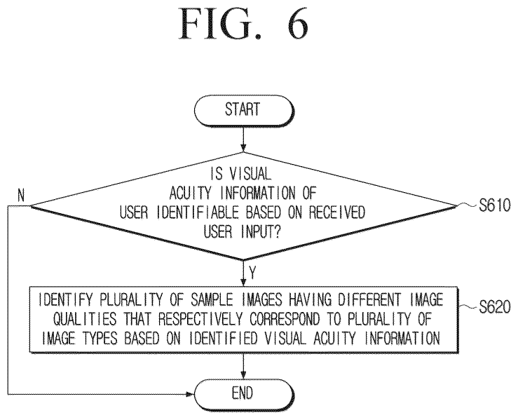

For example, the processor 140 may sequentially display the contrast sensitivity test image and the contour sensitivity test image. For example, the processor 140 may first display the contrast sensitivity test image on the display 110, and then display the contour sensitivity test image if a user input for the contrast sensitivity test image is received. This configuration is described in detail with reference to FIGS. 4A and 4B.

The processor 140 according to an embodiment may receive a user input for identifying the visual acuity information of the user.

Here, the visual acuity information of the user indicates information about the visual acuity level of the user, and for example, the visual acuity information may include information about the contrast sensitivity ability. For example, the processor 140 may identify a contrast sensitivity level corresponding to a first contrast sensitivity test image as the visual acuity information of the user if the first contrast sensitivity test image corresponding to the limit contrast sensitivity of the user is identified from the plurality of contrast sensitivity test images corresponding to the plurality of contrast sensitivity levels. In this case, the processor 140 may include information about a contour sensitivity level corresponding to a first contour sensitivity test image in the visual acuity information of the user if the first contour sensitivity test image corresponding to the limit contour sensitivity is identified from the contour sensitivity test images for the plurality of contour sensitivity levels, each corresponding to the first contrast sensitivity test image. That is, the contour sensitivity test may be performed based on the limit contrast sensitivity of the user, and refers to a test to identify the optimal contour sensitivity enabling for the user to efficiently watch an image within the limit contrast sensitivity of the user.

For example, the processor 140 may receive the user input for identifying the visual acuity information of the user through the user interface 120 if the contrast sensitivity test image and the contour sensitivity test image are displayed on the display 110. For example, the processor 140 assumes that the contrast sensitivity test image is displayed. The processor 140 may identify the contrast sensitivity level of the user based on a received user input if the processor 140 receives the user input for identifying the limit contrast sensitivity of the user. Alternatively, the processor 140 may identify the contour sensitivity level of the user based on a user input for identifying the limit contour sensitivity of the user if the contour sensitivity test image corresponding to an identified limit contrast sensitivity of the user is displayed after the limit contrast sensitivity of the user is identified.

The processor 140 according to an embodiment may identify the plurality of sample images having different image qualities based on the identified visual acuity information. For example, the processor 140 may identify the plurality of sample images having different image qualities respectively corresponding to a plurality of image types based on the identified visual acuity information if the visual acuity information of the user is identified based on the received user input.

For example, assume that the contrast sensitivity of the user is identified as a first contrast sensitivity and the contour contrast sensitivity of the user is identified as a first contour sensitivity. The T processor 140 may identify the plurality of sample images corresponding to the identified first contrast sensitivity and the first contour sensitivity.

Here, the plurality of sample images indicate images identified based on the identified visual acuity information, and for example, the plurality of sample images may include a plurality of images of different types. For example, the plurality of sample images may include images of a face type, a building type, a natural scene type, and a graphic type. Here, the image of the graphic type indicates an image including a different type of object including a shape or a pattern. Meanwhile, the plurality of sample images may have different image qualities. For example, the processor 140 may provide the sample images having the plurality of different image qualities on the display 110, and provide the user with the image having the image quality preferred by the user by selecting at least one of the plurality of provided sample images. A specific method for identifying the plurality of sample images having different image qualities is described in detail with reference to FIG. 6.

The processor 140 according to an embodiment may identify the image quality information corresponding to each of the plurality of image types based on the user input for selecting at least one of the plurality of sample images if the plurality of sample images are displayed on the display 110. Here, the image quality information indicates information about a plurality of items included in the image quality of an image, including color, contrast, and sharpness. For example, the image quality information corresponding to each of the plurality of sample images may be mapped to the sample image.

For example, the processor 140 may provide, on the display 110, the plurality of sample images corresponding to a first type among a plurality of types. The processor 140 may identify the sample image corresponding to the first type based on a received user input if the processor 140 receives the user input for identifying one of the plurality of sample images corresponding to the first type. The processor 140 may identify the image quality information of the sample image corresponding to the identified first type from the plurality of sample images as the image quality information of the first type. Accordingly, the processor 140 may identify the image quality information of an image having the image quality preferred by the user at the current visual acuity level of the user.

The processor 140 according to an embodiment may display an image on the display 110 based on the identified image quality information. For example, assume that the image quality information corresponding to the first type is identified. The processor 140 may identify a type of an image provided on the display 110, and change an image quality of the image based on the image quality information corresponding to the identified type. The processor 140 may display an image having the changed image quality on the display 110. This configuration is described in detail with reference to FIG. 7.

According to the above-described example, the display apparatus 100 may obtain the visual acuity information of the user through the contrast sensitivity test and the contour sensitivity test, provide the user with the sample image for identifying the image quality preferred by the user based on the obtained visual acuity information, and identify the image quality information preferred by the user based on the obtained sample image. The display apparatus 100 may provide the image reflecting the image quality preferred by the user based on the identified image quality information, and provide user with the image reflecting the visual acuity level of the user and the user preference.

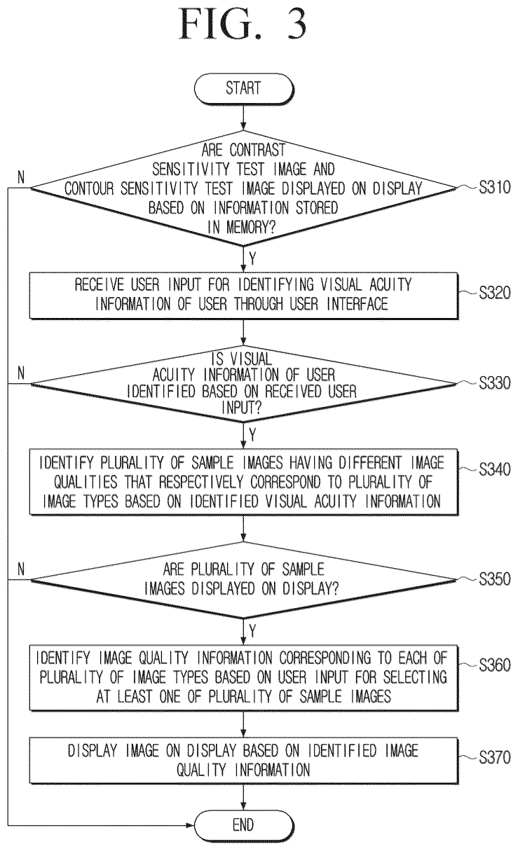

FIG. 3 is a flowchart for describing the control method for a display apparatus according to an embodiment.

Referring to FIG. 3, the control method according to an embodiment may include identifying whether the contrast sensitivity test image and the contour sensitivity test image are displayed on the display 110 based on information stored in the memory 130 (S310).

For example, the processor 140 may first display the contrast sensitivity test image on the display 110. For example, the processor 140 may display the contrast sensitivity test image as shown in FIG. 1, and is not limited thereto, and the processor 140 may also sequentially display images respectively corresponding to the plurality of contrast sensitivity levels. Next, the processor 140 may display the contour sensitivity test image on the display 110. For example, the processor 140 may identify the contour sensitivity test image corresponding to an identified limit contrast sensitivity level based on the information stored in the memory 130, and display the same on the display 110 if the limit contrast sensitivity level of the user is identified as the contrast sensitivity test image is displayed.

Next, according to an embodiment, the control method may include receiving the user input for identifying the visual acuity information of the user through the user interface 120 (S320) if the contrast sensitivity test image and the contour sensitivity test image are displayed (S310: Y). For example, the processor 140 may first receive a user input for identifying the limit contrast sensitivity level of the user through the user interface 120 if the contrast sensitivity test image is displayed on the display 110. Next, if the limit contrast sensitivity level of the user is identified, the processor 140 may receive a user input for identifying a limit contour sensitivity level of the user if the contour sensitivity test image is displayed on the display 110.

Next, the control method according to an embodiment may include identifying whether the visual acuity information of the user is identified based on the received user input (S330). For example, the processor 140 may receive the user input for identifying the limit contrast sensitivity level of the user and the limit contour sensitivity level of the user through the user interface 120, and identify the limit contrast sensitivity level and the limit contour sensitivity level as the visual acuity information of the user.

Next, the control method according to an embodiment may include identifying the plurality of sample images having different image qualities that respectively correspond to the plurality of image types based on the identified visual acuity information (S340) if the visual acuity information of the user is identified (S330: Y). For example, the processor 140 may identify the plurality of sample images corresponding to the identified visual acuity information if the visual acuity information including the limit contrast sensitivity level and limit contour sensitivity level of the user is identified. In this case, the plurality of sample images may include the plurality of sample images having different image qualities for each of the plurality of types. For example, the memory 130 may store the plurality of sample images having different image qualities for each of the plurality of types corresponding to the visual acuity level of the user, and the processor 140 may identify the plurality of sample images for each of the plurality of types corresponding to the identified visual acuity information based on the information stored in the memory 130. However, the present disclosure is not limited thereto, and this configuration is described in detail with reference to FIG. 6.

Next, the control method according to an embodiment may include identifying whether the plurality of sample images are displayed on the display 110 (S350). For example, the processor 140 may display the plurality of sample images for each of the plurality of types on the display 110. In this case, for example, the processor 140 may display the plurality of sample images corresponding to the first type among the plurality of types on one screen. However, the present disclosure is not limited thereto, and the processor 140 may also sequentially display the plurality of sample images corresponding to the first type among the plurality of types based on the contour contrast sensitivity.

Next, the control method according to an embodiment may include identifying the image quality information corresponding to each of the plurality of image types based on the user input for selecting at least one of the plurality of sample images (S360) if the plurality of sample images are displayed on the display 110 (S350: Y). For example, the processor 140 may identify the image quality information corresponding to the sample image identified based on the user input among the plurality of sample images as the image quality information corresponding to each of the plurality of image types. For example, the processor 140 may identify the image quality information corresponding to the identified sample image as the image quality information corresponding to the first type if the sample image corresponding to the first type among the plurality of image types is identified based on the user input.

Next, the control method according to an embodiment may include displaying an image on the display 110 based on the identified image quality information (S370). For example, the processor 140 may identify the image reflecting the identified image quality information, and provide the identified image on the display 110 if the image quality information is identified based on the user input. For example, assume that the image quality information corresponding to a person type among the plurality of types is identified. The processor 140 may change the image quality of the identified image based on the identified image quality information, and display the image having the changed image quality on the display 110 if an image corresponding to the person type among the images displayed on the display 110 is identified. This configuration is described in detail with reference to FIG. 7.

FIGS. 4A and 4B are diagrams for describing a method for identifying the visual acuity information according to an embodiment.

Referring to FIG. 4A, the control method according to an embodiment may include identifying whether the contrast sensitivity test image is displayed on the display 110 (S410). For example, the processor may first display the contrast sensitivity test image on the display 110 based on the information stored in the memory 130.

Next, the control method according to an embodiment may include identifying first visual acuity information corresponding to the contrast sensitivity of the user based on the user input received through the user interface 120 (S420) if the contrast sensitivity test image is displayed (S410: Y). Here, the first visual acuity information indicates a magnitude of the limit contrast sensitivity of the user.

For example, assume that the plurality of contrast sensitivity test images corresponding to the plurality of contrast sensitivity levels are displayed on the display 110. The processor 140 may identify, as the first visual acuity information of the user, the magnitude of the contrast sensitivity level corresponding to the first contrast sensitivity test image if the first contrast sensitivity test image corresponding to the limit contrast sensitivity of the user is identified from the plurality of contrast sensitivity test images corresponding to the plurality of contrast sensitivity levels. That is, the processor 140 may identify, as the first visual acuity information of the user, the magnitude of the limit contrast sensitivity level, which is a maximum value of the contrast sensitivity identifiable by the user.

Next, the control method according to an embodiment may include identifying whether the contour sensitivity test image corresponding to the first visual acuity information is identified based on the identified first visual acuity information (S430). For example, the memory 130 may store the plurality of contour sensitivity test images respectively corresponding to the plurality of contrast sensitivity levels, and the plurality of contour sensitivity test images may correspond to different contour sensitivities. For example, as shown in FIG. 1, images corresponding to different contour sensitivities may be displayed on one screen. However, the present disclosure is not limited thereto, and the images respectively corresponding to different contour sensitivities may be sequentially displayed on the screen. The processor 140 may identify the plurality of contour sensitivity test images corresponding to the identified first visual acuity information based on the information stored in the memory.

Next, the control method according to an embodiment may include providing the contour sensitivity test image corresponding to the first visual acuity information on the display (S440) if the contour sensitivity test image corresponding to the first visual acuity information is identified (S430: Y). For example, the processor 140 may display the plurality of contour sensitivity test images corresponding to the identified first visual acuity information on the display 110. In this case, as shown in FIG. 1, the plurality of images having different contour sensitivities may be displayed on one screen. However, the present disclosure is not limited thereto. For example, the plurality of images having different contour sensitivities may be sequentially displayed on the screen based on the magnitude of the contour sensitivity. For example, after a first image having the first contour sensitivity is displayed, a second image having a second contour sensitivity may be sequentially displayed.

Next, the control method according to an embodiment may include identifying whether the contour sensitivity test image corresponding to the first visual acuity information is displayed (S450).

Next, the control method according to an embodiment may include identifying second visual acuity information corresponding to the contour sensitivity of the user based on the user input received through the user interface 120 (S460) if the contour sensitivity test image corresponding to the first visual acuity information is displayed (S450: Y). Here, the second visual acuity information indicates a magnitude of the limit contour sensitivity level of the user among the plurality of contour sensitivities. For example, the processor 140 may identify, as the second visual acuity information of the user, the magnitude of the limit contour sensitivity level, which is a maximum value of the contour sensitivity identifiable by the user among the plurality of contour sensitivity levels corresponding to the limit contrast sensitivity of the user. That is, the processor 140 may identify the contour contrast sensitivity level in the image reflecting the limit contrast sensitivity of the user.

For example, the processor 140 may identify the second visual acuity information corresponding to the limit contour sensitivity level of the user among the plurality of contour sensitivity levels based on the user input received through the user interface 120 if the contour sensitivity test image corresponding to the first visual acuity information is displayed. This configuration is described in more detail below with reference to FIG. 4B.

Referring to FIG. 4B, the processor 140 according to an embodiment may display the contrast sensitivity test image and the contour sensitivity test image on the display 110, and identify the visual acuity information of the user based on the received user input. For example, the processor 140 may perform at least one test to measure the visual acuity level of the user.

For example, the processor 140 may perform a plurality of tests 400 to measure the visual acuity level of the user. For example, seven (CL1 to CL7) contrast sensitivity levels and three (eL1 to eL3) contour sensitivity levels (or edge enhancement levels) may be provided to measure the visual acuity level of the user.

The processor 140 may first perform a first test corresponding to a first sequence. The processor 140 may display, on the display 110, a contrast sensitivity test image corresponding to the CL1 level, which has a relatively lowest contrast sensitivity level, and receive the user input based thereon.

Next, if the contrast sensitivity test image corresponding to the CL1 level is displayed, the processor 140 may perform a second test corresponding to a second sequence without displaying an image for measuring the contour sensitivity level if the user input indicating that the image is identifiable (or correct) is received. The processor 140 may display the contrast sensitivity test image corresponding to the CL2 level on the display 110 without displaying the image corresponding to the CL1 level based on history information of the first sequence.

Next, the processor 140 may perform a third test corresponding to a third sequence without displaying the image for measuring the contour sensitivity level if the user input indicating that the image is identifiable is received if the contrast sensitivity test image corresponding to the CL2 level is displayed. The processor 140 may display the contrast sensitivity test image corresponding to the CL3 level on the display 110 without displaying the images corresponding to the CL1 and CL2 levels based on history information of the first and second sequences.

Next, the processor 140 may display the contour sensitivity test image corresponding to the eL1 level on the display 110 if the contrast sensitivity test image corresponding to the CL3 level is displayed and the user input indicating that the displayed image is unidentifiable (or incorrect) is received. In this case, the contour sensitivity test image corresponding to the eL1 level may be an image having the contrast sensitivity corresponding to the CL3 level. That is, the image having the contour sensitivity of the eL1 level and the contrast sensitivity of the CL3 level may be displayed on the display 110. As the contour sensitivity test image corresponding to the eL1 level is displayed, the processor 140 may perform a fourth test corresponding to a fourth sequence if the user input corresponding to a correct answer is received.

The processor 140 may display the contrast sensitivity test image corresponding to the CL4 level on the display 110 instead of displaying the image corresponding to the CL1 to CL3 levels based on history information of the first to third sequences. In this case, a process of displaying the image corresponding to the contrast sensitivity level (CL3), which is identifiable by the user due to the contour sensitivity level corresponding to the eL1 level, is omitted.

The processor 140 may display the contour sensitivity test image corresponding to the eL1 level on the display 110 if the user input indicating that the contrast sensitivity test image corresponding to the CL4 level is identifiable is received. In this case, the contour sensitivity test image corresponding to the eL1 level may be an image having the contrast sensitivity corresponding to the CL4 level. The processor 140 may perform a fifth test corresponding to a fifth sequence if the user input indicating that the contour sensitivity test image corresponding to the eL1 level is identifiable is received.

The processor 140 may then perform the plurality of tests including sixth and seventh sequences. The processor 140 may identify the visual acuity information of the user by using the first to seventh sequences. For example, the visual acuity information of the user may include the visual acuity information corresponding to each of the third to seventh sequences. For example, the first visual acuity information (CL3) corresponding to the third sequence and the second visual acuity information (eL1) corresponding thereto may be included in the visual acuity information. In addition, the first visual acuity information (CL4) corresponding to the fourth sequence and the second visual acuity information (eL1) corresponding thereto may be included in the visual acuity information. Similarly, the first visual acuity information (cL7) corresponding to the seventh sequence and the second visual acuity information (eL3) corresponding thereto may be included in the visual acuity information. That is, the visual acuity information may include data corresponding to the plurality of sequences.

Meanwhile, the test shown in FIG. 4B is only an embodiment, and the processor 140 may execute different tests from the test shown in FIG. 4B. For example, the number of contrast sensitivity levels may be less than or more than seven, and the number of contour sensitivity levels may also be less than or more than three. In addition, test sequences different from the sequences shown in FIG. 4B may be performed.

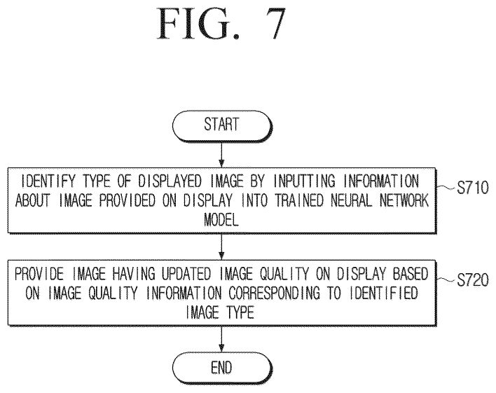

FIG. 5 is a diagram for describing a method for identifying the visual acuity information according to an embodiment.

Referring to FIG. 5, the control method according to an embodiment may first include displaying, on the display 110, the plurality of contrast sensitivity test images respectively corresponding to the plurality of contrast sensitivity levels and the contour sensitivity test images having different contour sensitivities that respectively correspond to the plurality of contrast sensitivity test images (S510). For example, the memory 130 may include the plurality of contrast sensitivity test images respectively corresponding to the plurality of contrast sensitivity levels and the contour sensitivity test images having different contour sensitivities that respectively correspond to the plurality of contrast sensitivity test images. For example, the contrast sensitivity test image corresponding to the first contrast sensitivity level may be stored in the memory 130, and the image for each of the plurality of contour sensitivity levels that corresponds to the contrast sensitivity test image corresponding to the first contrast sensitivity level may also be stored in the memory 130.

Next, the control method according to an embodiment may include identifying whether the contrast sensitivity of the first contrast sensitivity test image is unidentifiable from the plurality of contrast sensitivity test images based on the user input received through the user interface 120 (S520).

Next, the control method according to an embodiment may include identifying the contrast sensitivity level corresponding to the first contrast sensitivity test image as the first visual acuity information corresponding to the contrast sensitivity of the user based on the information stored in the memory 130 (S530) if the contrast sensitivity of the first contrast sensitivity test image is identified as unidentifiable (S520: Y).

Next, the control method according to an embodiment may include identifying whether at least one contour sensitivity test image corresponding to the first visual acuity information is displayed based on the information stored in the memory 130 (S540).

Next, the control method according to an embodiment may include identifying the second visual acuity information corresponding to the contour sensitivity of the user based on the user input received through the user interface 120 (S550) if at least one contour sensitivity test image corresponding to the first visual acuity information is displayed (S540: Y). For example, the processor 140 may display the contour sensitivity test image having a different contour level that corresponds to the first visual acuity information. After the contour sensitivity test image having a different contour level is displayed, the processor 140 may identify the second visual acuity information of the user based on the received user input.

FIG. 6 is a diagram for describing a method for identifying the plurality of sample images and the image quality information according to an embodiment.

Referring to FIG. 6, the control method according to an embodiment may include identifying whether the visual acuity information of the user is identifiable based on the received user input (S610). For example, the processor 140 may display the contrast sensitivity test image and the contour sensitivity test image on the display 110, and identify the visual acuity information including the first visual acuity information and the second visual acuity information based on the user input corresponding thereto.

Next, the control method according to an embodiment may include identifying the plurality of sample images having different image qualities that respectively correspond to the plurality of image types based on the identified visual acuity information (S620) if the visual acuity information of the user is identified (S610: Y). Here, the plurality of image types may include the face type, the building type, the natural scene type, and the graphic type. Here, the face type image indicates an image including a human face in the image, and the building type image indicates an image including a building among the objects in the image. The natural scene type image indicates an image including a natural scene in the image, and the graphic type image indicates an image including at least one of a shape, a letter, and a pattern in the image.

For example, the memory 130 may further include the plurality of sample images having different image qualities that respectively correspond to the plurality of image types mapped to the visual acuity information of the user. For example, the memory 130 may store the plurality of sample images having different image qualities that correspond to the visual acuity information corresponding to the CL3 level and the eL1 level, in which case the plurality of sample images for each of the plurality of image types may be stored, respectively. Alternatively, the memory 130 may store the plurality of sample images having different image qualities that correspond to the visual acuity information corresponding to the CL4 level and the eL1 level. Meanwhile, for example, the processor 140 may identify the plurality of sample images having different image qualities that respectively correspond to the face type, the building type, the natural scene type, and the graphic type based on the information stored in the memory 130 if the visual acuity information of the user is identified based on the received user input.

For example, if the visual acuity information of the user is identified as corresponding to the CL3 level and the eL1 level based on the received user input, the processor 140 may identify the plurality of sample images of the person type having different image qualities, the plurality of sample images of the building type having different image qualities, the plurality of sample images of the natural scene type having different image qualities, and the plurality of sample images of the graphic type having different image qualities, which correspond to the CL3 level and the eL1 level, based on the information stored in the memory 130.

Meanwhile, for example, the memory 130 may store a function corresponding to a correlation between the visual acuity information of the user and the image quality of an image. Here, the function corresponding to the correlation between the visual acuity information of the user and the image quality of an image refers to a function trained to output a value of an image quality setting feature (e.g., REGISTER zero and 1) of an image corresponding to the user if data about user context information and the visual acuity information of the user are input. For example, the function corresponding to the correlation between the visual acuity information of the user and the image quality of an image may be trained using the data of the user context information, the visual acuity information of the user, and information about the user preference for the plurality of sample images (e.g., information about the image quality of the sample image preferred by the user among the plurality of sample images). Here, the user context information may include at least one of a user age, a user gender, a type of disability the user has, and a period of time the user experiences the disability.

For example, the function corresponding to the correlation may be used to identify the plurality of sample images respectively corresponding to the plurality of image types and the image quality information corresponding to each of the plurality of sample images if the user context information and the visual acuity information of the user are input into the function. Alternatively, for example, the function corresponding to the correlation may be used to identify a probability value of each of the plurality of sample images respectively corresponding to the plurality of image types. Here, the probability value of each of the plurality of sample images indicates a probability value for selecting the first sample image within the first type. Meanwhile, for example, the image quality information corresponding to each of the plurality of sample images may include at least one of a contrast enhancement value, a sharpness enhancement value, a gamma adjustment value, or a color adaption value.

For example, the processor 140 may input the user context information and the identified visual acuity information of the user, received through a communication interface, into the function to identify the plurality of sample images having different image qualities that respectively correspond to the plurality of image types and the image quality information corresponding to each of the plurality of sample images having different image qualities. For example, if the user context information is received and the visual acuity information of the user corresponding to the plurality of sequences is identified, the processor 140 may input the same into the function to identify the image quality information corresponding to each of the plurality of sample images having different image qualities that correspond to the first image type and to identify the image quality information corresponding to each of the plurality of sample images having different image qualities that correspond to a second image type.

Here, for example, the processor 140 may receive the user context information from the external apparatus through the communication interface. However, the present disclosure is not limited thereto, and the processor 140 may also receive the user input corresponding to the user context information through the user interface 120.

According to an embodiment, the processor 140 may identify the image quality information corresponding to a selected at least one sample image as the image quality information corresponding to each of the plurality of image types if the user input is received through the user interface 120 for selecting at least one of the plurality of sample images. For example, the processor 140 may identify the image quality information corresponding to each of the selected sample images corresponding to each of the plurality of image types as the image quality information corresponding to each of the plurality of image types if the user input is received through the user interface 120 for selecting one of the sample images respectively corresponding to the plurality of image types.

For example, if the processor 140 receives the user input for selecting a first sample image among the plurality of sample images having different image qualities that correspond to the first type (e.g., the face type), the processor 140 may identify the image quality information corresponding to the first sample image as the image quality information corresponding to the first type. Alternatively, for example, the processor 140 may identify the image quality information corresponding to the second sample image as the image quality information corresponding to a second type if the processor 140 receives the user input for selecting a second sample image among the plurality of sample images having different image qualities corresponding to the second type (e.g., the building type).

FIG. 7 is a diagram for describing a method for providing an updated image by using a neural network model according to an embodiment.

Referring to FIG. 7, the control method according to an embodiment may include identifying the type of the displayed image by inputting the information about an image provided on the display 110 into the trained neural network model (S710). For example, the memory 130 may store the neural network model trained to classify the type of the image displayed on the display apparatus 100. For example, the trained neural network model may be an input image classifier that classifies images. The trained neural network model may identify whether the type of an input image is the natural scene type, a city type, the person type, or the graphic type, is not limited thereto, and may identify whether the type of an input image is a different type of image other than the above-described types. For example, the trained neural network model may be the neural network model trained to output the type of the input image if an image currently provided on the display 110 or an image to be provided on the display 110 is input thereinto. For example, the processor 140 may identify the type of the provided image by inputting the image provided on the display 110 into the trained neural network model.

Next, the control method according to an embodiment may include providing the image having an updated image quality on the display 110 based on the image quality information corresponding to the identified image type (S720). For example, assume that the image quality information corresponding to each of the plurality of image types is identified. The processor 140 may input the image provided on the display 110 into the trained neural network model to identify the type of the image. If the image is identified as belonging to the first type, the processor 140 may update the image based on the image quality information corresponding to the first type. For example, the processor 140 may update the image provided on the display 110 to have the image quality of the first type preferred by the user, and display the updated image on the display 110.

However, the present disclosure is not limited thereto, and for example, if the image quality information is identified, the processor 140 may transmit the image quality information to the external apparatus through the communication interface (not shown), and may receive the updated image from the external apparatus (not shown). For example, if the external apparatus is implemented as an image rendering apparatus, the external apparatus may identify the image having updated quality information, and the processor 140 may receive the image having the updated image quality information from the external apparatus through the communication interface (not shown).

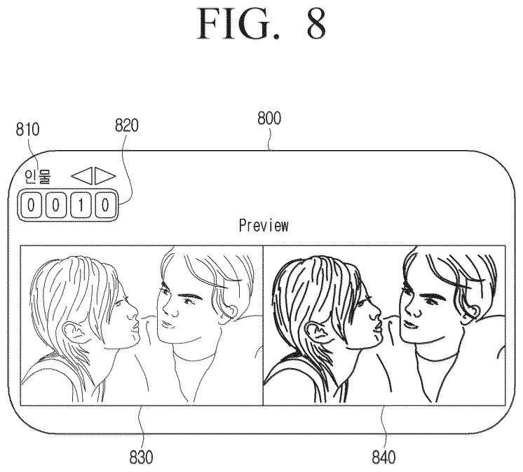

FIG. 8 is a diagram for describing a method for receiving the user input for selecting at least one of the plurality of sample images according to an embodiment.

Referring to FIG. 8, the processor 140 according to an embodiment may provide, on the display 110, a user interface (UI) 800 for selecting at least one of the plurality of sample images. For example, the UI may display the plurality of sample images, and the plurality of sample images may include a reference sample image 830 and a comparison sample image 840 having a different image quality from the reference sample image 830. For example, the processor 140 may identify the plurality of sample images having different image qualities that respectively correspond to the plurality of image types based on the information stored in the memory 130, and provide the UI 800 including the plurality of identified sample images. Alternatively, for example, the processor 140 may identify the plurality of sample images by using the function corresponding to the correlation between the visual acuity information of the user and the image quality of an image, and provide the UI 800 including the same.

Meanwhile, for example, the UI 800 may include a type 810 corresponding to the sample image and an identification number 820 (or a sensitivity code) of the comparison sample image 840. Here, the sensitivity code indicates code information assigned to each of the plurality of sample images. For example, as shown in FIG. 8, if the sample image of the person type is provided, the identification numbers 820 corresponding to the plurality of sample images may be provided through the UI. In addition, if the sample images of the natural scene type, the building type, and the graphic type are provided, the plurality of sample images corresponding to the respective types and the identification numbers corresponding thereto may be provided through the UI.

For example, if the user interface (UI) 800 for selecting at least one of the plurality of sample images is provided and a user input is received through the user interface 120 for selecting one of the plurality of sample images corresponding to the first type, the processor 140 may identify one image corresponding to the first type based on the received user input, and identify the image quality information corresponding to the identified image as the image quality information corresponding to the first type.

According to the above-described example, the display apparatus 100 may provide the plurality of sample images to the user based on the visual acuity information of the user identified through the contrast sensitivity test and the contour sensitivity test, identify the image quality information corresponding to each of the plurality of types based on the user input for selecting any one of the plurality of sample images, and provide the image having the updated image quality to the user by using the identified image quality information. Accordingly, the display apparatus 100 may provide the user with the image reflecting an objective visual acuity level of the user and the image quality information of a subjectively preferred image preferred by the user.

Meanwhile, for example, the display apparatus 100 may also provide guide information to guide a user selection. Here, the guide information may be guide information for identifying a score of the sample image, for example, “Please rate how well you may identify the following image” or “Which of the following four images do you prefer the most?”

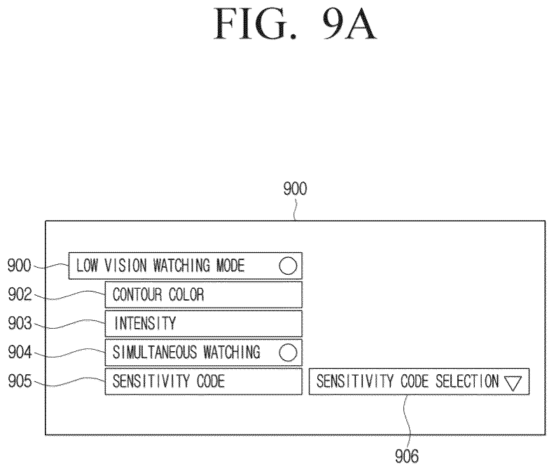

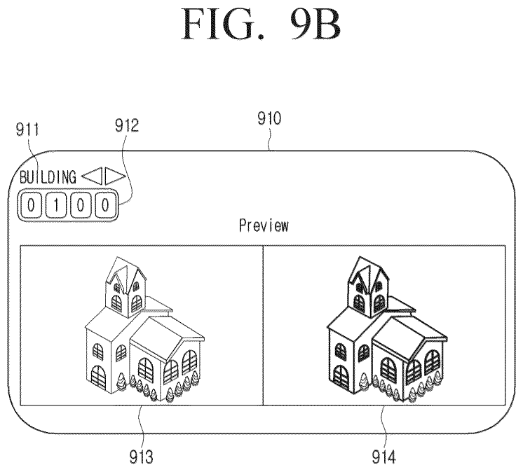

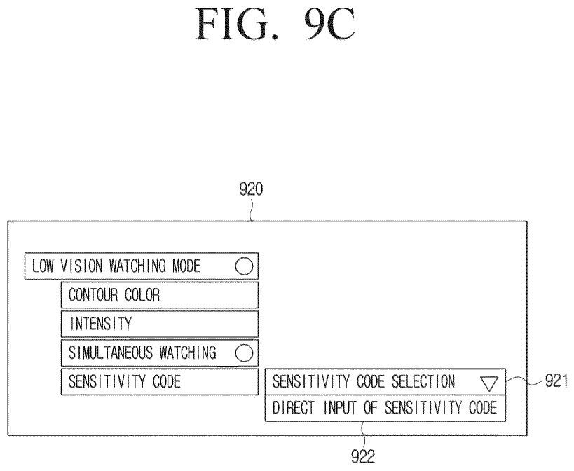

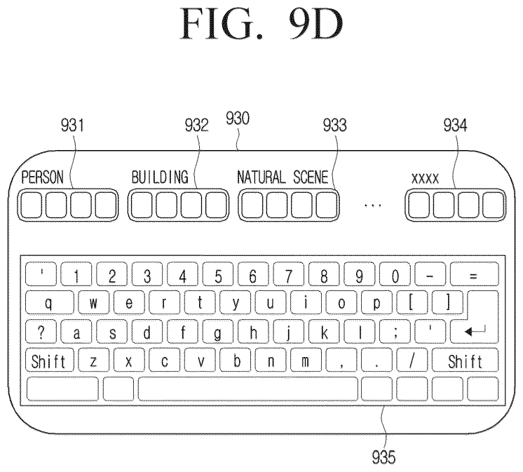

FIGS. 9A to 9D are diagrams for describing a method for providing a user interface (UI) according to an embodiment.

Referring to FIG. 9A, the processor 140 according to an embodiment may display a UI 900 for performing a plurality of functions on the display 110. For example, the UI 900 for guiding an assistive function for a low vision user may be displayed if a watching mode 901 for the low vision user is executed, and the UI 900 may include a UI corresponding to a contour color selection function 902, an intensity selection function 903, a simultaneous watching mode selection function 904, a sensitivity code function 905, and a sensitivity code selection function 906. Here, the contour color selection function 902 refers to a function for identifying a contour color included in an image. The intensity selection function 903 refers to a function for selecting an intensity (or strength) of the image quality of an image, including the brightness, contrast, and line brightness of an image. The simultaneous watching mode selection function 904 refers to a function for simultaneously displaying, on the display 110, the image having the updated image quality and the image whose image quality is not updated (or an image for a general user who is not the low vision user). The sensitivity code function 905 refers to a function for enabling the user to select a desired level of image quality. If the sensitivity code corresponding to the sample image selected by the user among the plurality of sample images corresponding to the first type is selected, the processor 140 may perform image quality processing by using the selected sensitivity code. For example, if “0100” indicates the sensitivity code corresponding to the sample image selected by the user among the plurality of sample images corresponding to the first type, the processor 140 may identify the image quality information corresponding to the sensitivity code “0100” and update the image based thereon.

Referring to FIG. 9B, the processor 140 according to an embodiment may provide a UI 910 for selecting at least one of the plurality of sample images on the display 110. For example, the UI 910 for selecting at least one of the plurality of sample images may include a type 911 corresponding to the sample image, a reference sample image 913, a comparison sample image 914, and a sensitivity code 912 corresponding to the comparison sample image 914.

For example, the processor 140 may provide, on the display 110, a UI including the plurality of sample images corresponding to the first type among the plurality of image types. Next, if a user input is received through the user interface 120 for selecting the first image among the plurality of sample images corresponding to the first type, the processor 140 may identify the image quality information corresponding to the first image as the image quality information corresponding to the first type. Next, the processor 140 may provide, on the display 110, a UI including the plurality of sample images corresponding to the second type among the plurality of image types. Next, if a user input is received through the user interface 120 for selecting the second image among the plurality of sample images corresponding to the second type, the processor 140 may identify the image quality information corresponding to the second image as the image quality information corresponding to the second type. Accordingly, the display apparatus 100 may identify the image quality information corresponding to each of the plurality of types.

Referring to FIG. 9C, the processor 140 according to an embodiment may display a UI 920 on the display 110 to receive the sensitivity code corresponding to each of the plurality of types. For example, the UI 920 may include a UI 922 to directly receive the sensitivity code from the user. For example, the processor 140 may receive, from the user, a user input for performing a function for directly inputting the sensitivity code among the plurality of functions included in sensitivity code selection 921, and may receive, from the user, the sensitivity code corresponding to the type of the image.

Referring to FIG. 9D, if the user input is received through the UI 922 for directly receiving the sensitivity code, the processor 140 according to an embodiment may display, on the display 110, a UI 930 for directly receiving the sensitivity code corresponding to each of the plurality of types. For example, the UI 930 may include a UI 931 for receiving a sensitivity code of the person type among the plurality of types, a UI 932 for receiving a sensitivity code of the building type, a UI 933 for receiving a sensitivity code of the natural scene type, and a UI 934 for receiving a sensitivity code of an Nth type. In addition, the UI 930 may include a function 935 corresponding to a virtual keyboard for receiving the user input. The processor 140 may identify the image quality information corresponding to the received sensitivity code and update the image based on the identified image quality information.

Meanwhile, for example, the identified sensitivity code may be identified as a sensitivity code corresponding to the user and stored in the memory 130. For example, if “001” is a sensitivity code corresponding to the face type of a first user, “010” is a sensitivity code corresponding to the building, “001” is a sensitivity code corresponding to the natural scene, and “100” indicates a sensitivity code corresponding to the graphic, the processor 140 may identify the sensitivity code corresponding to the first user as “USER 1_FC001_BD010_NT001_GP100” and store the same in the memory 130. For example, if the processor 140 receives a request signal for the image quality information corresponding to the first user from the external apparatus (not shown), the sensitivity code corresponding to the first user may be transmitted to the external apparatus (not shown) through the communication interface (not shown). Accordingly, the external apparatus (not shown) may provide a customized image to the user without having to separately measure the visual acuity level of the user or perform a user preference survey.

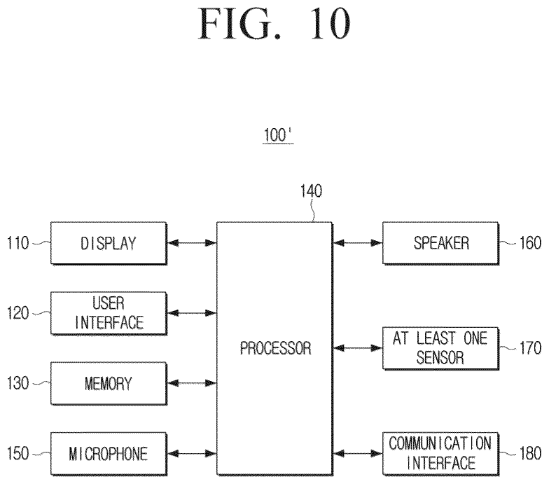

FIG. 10 is a block diagram showing a specific configuration of a display apparatus according to an embodiment.

Referring to FIG. 10, a display apparatus 100′ may include the display 110, the user interface 120, the memory 130, at least one processor 140, a microphone 150, a speaker 160, at least one sensor 170, and a communication interface 180. Detailed descriptions of the components shown in FIG. 10 that overlap with the components shown in FIG. 2 are omitted.

The microphone 150 may indicate a module that obtains sound and converts the same into an electric signal, and may be a condenser microphone, a ribbon microphone, a moving coil microphone, a piezoelectric element microphone, a carbon microphone, a micro electro mechanical system (MEMS) microphone. In addition, the microphone 150 may be implemented in an omnidirectional, bidirectional, unidirectional, subcardioid, supercardioid, or hypercardioid manner.

Various embodiments may be provided in which the display apparatus 100′ performs an operation corresponding to a user voice signal received through the microphone 150.

The speaker 160 may include a tweeter for reproducing high-frequency sounds, a midrange for reproducing medium-frequency sounds, a woofer for reproducing low-frequency sounds, a subwoofer for reproducing ultra-low-frequency sounds, an enclosure for controlling resonance, a crossover network for dividing a frequency of an electric signal input into the speaker into bands, or the like.

The speaker 160 may output an audio signal to the outside of the display apparatus 100′. The speaker 160 may output multimedia reproduction, recording reproduction, various notification sounds, voice messages, or the like. The display apparatus 100′ may include an audio output apparatus such as the speaker 160, and may include an output apparatus such as an audio output terminal. In particular, the speaker 160 may provide obtained information, information processed and produced based on the obtained information, a response result to a user voice, or an operation result, or the like in a voice form.

At least one sensor 170 (hereinafter referred to as the sensor) may include a plurality of sensors of various types. The sensor 170 may measure a physical quantity or detect an operating state of the display apparatus 100′, and convert the measured or detected information into the electrical signal. The sensor 170 may include a camera, and the camera may include a lens that focuses visible light or other optical signals reflected from an object and received onto an image sensor, and the image sensor capable of detecting visible light or other optical signals. Here, the image sensor may include a two-dimensional (2D) pixel array divided into a plurality of pixels.

At least one sensor 170 according to an embodiment may include at least one of an impedance sensor, a photoplethysmography (PPG) sensor, an electrodemal activity (EDA) sensor, a temperature sensor, or an acceleration sensor. The PPG sensor refers to a sensor that measures an amount of blood flowing in a blood vessel by using an optical characteristic of the skin to determine a heart rate activity state. The EDA sensor refers to a sensor that provides information about an autonomic nervous system of a human body by coupling with the heart rate, respiration rate, blood pressure, or the like of a human. The processor 140 may obtain body hydration data of the user based on sensing information received from at least one of the impedance sensor, the photoplethysmography (PPG) sensor, the electrodemal activity (EDA) sensor, or the temperature sensor by using a predetermined algorithm.

The communication interface 180 may input and output various types of data. For example, the communication interface 180 may transmit and receive the various types of data to and from the external apparatus (e.g., a source apparatus), an external storage medium (e.g., the universal serial bus (USB) memory), an external server (e.g., a web hard) by using a communication method such as access point (AP)-based wireless fidelity (Wi-Fi, wireless local area network (LAN)), Bluetooth, Zigbee, wired/wireless local area network (LAN), wide area network (WAN), Ethernet, IEEE 1394, high definition multimedia interface (HDMI), universal serial bus (USB), mobile high-definition link (MHL), audio engineering society/European broadcasting union (AES/EBU) communication, optical communication, or coaxial communication.

For example, the communication interface 180 may include a Bluetooth low energy (BLE) module. The BLE refers to a Bluetooth technology that enables transmission and reception of low-power and low-capacity data in a 2.4 gigahertz (GHz) frequency band having a range of about 10 m. However, the present disclosure is not limited thereto, and the communication interface 180 may include a Wi-Fi communication module. That is, the communication interface 180 may include at least one of the Bluetooth low energy (BLE) module or the Wi-Fi communication module.

For example, the communication interface 180 may use a different communication module to communicate with the external apparatus such as the remote control apparatus or the external server. For example, the communication interface 180 may use at least one of the Ethernet module or the Wi-Fi module to communicate with the external server, and may use the Bluetooth module to communicate with the external apparatus such as the remote control apparatus. However, this case is only an embodiment, and the communication interface 180 may use at least one communication module among various communication modules in case of communicating with the plurality of external apparatus or external servers.

According to the above-described example, the display apparatus 100′ may obtain the visual acuity information of the user through the contrast sensitivity test and the contour sensitivity test, provide the user with the sample image for identifying the image quality preferred by the user based on the obtained visual acuity information, and identify the image quality information preferred by the user based thereon. The display apparatus 100′ may provide the image reflecting the image quality preferred by the user based thereon, and provide the user with the image reflecting the visual acuity level of the user and the user preference.

Meanwhile, the methods according to the various embodiments of the present disclosure described above may be implemented in the form of an application capable of being installed on a conventional display apparatus. Alternatively, the methods according to the various embodiments of the present disclosure described above may be performed using a deep learning-based trained neural network (or deep trained neural network), that is, the learning network model. In addition, the methods according to the various embodiments of the present disclosure described above may be implemented only by software upgrade or hardware upgrade of the conventional display apparatus. In addition, the various embodiments of the present disclosure described above may be performed through an embedded server included in the display apparatus, or the external server of the display apparatus.

Meanwhile, according to an embodiment of the present disclosure, the various embodiments described above may be implemented in software including an instruction stored on a machine-readable storage medium (for example, a computer-readable storage medium). A machine may be a apparatus that invokes the stored instruction from a storage medium, may be operated based on the invoked instruction, and may include the display apparatus (e.g., display apparatus A) according to the disclosed embodiments. If the instruction is executed by the processor, the processor may directly perform a function corresponding to the instruction or other components may perform the function corresponding to the instruction under control of the processor. The instruction may include codes provided or executed by a compiler or an interpreter. The machine-readable storage medium may be provided in the form of a non-transitory storage medium. Here, the term “non-transitory” indicates that the storage medium is tangible without including a signal, and does not distinguish whether data are semi-permanently or temporarily stored on the storage medium.

In addition, according to an embodiment, the methods according to the various embodiments described above may be included and provided in a computer program product. The computer program product may be traded as a commodity between a seller and a purchaser. The computer program product may be distributed in a form of the machine-readable storage medium (for example, a compact disc read only memory (CD-ROM)), or may be distributed online through an application store (for example, PlayStore™. In case of the online distribution, at least a part of the computer program product may be at least temporarily stored or temporarily provided on a storage medium such as the memory of a manufacturer server, an application store server, or a relay server.

In addition, each of the components (for example, modules or programs) according to the various embodiments described above may include a single entity or a plurality of entities, and some of the corresponding sub-components described above may be omitted or other sub-components may be further included in the various embodiments. Alternatively or additionally, some of the components (for example, the modules or the programs) may be integrated into the single entity, and may perform functions performed by the respective corresponding components before being integrated in the same or similar manner. Operations performed by the modules, the programs, or other components according to the various embodiments may be executed in a sequential manner, a parallel manner, an iterative manner, or a heuristic manner, at least some of the operations may be performed in a different order or be omitted, or other operations may be added.

Although certain embodiments are shown and described in the present disclosure as above, the present disclosure is not limited to the above-described specific embodiments, and may be variously modified by those skilled in the art to which the present disclosure pertains without departing from the gist of the present disclosure as claimed in the accompanying claims. Accordingly, the scope of various embodiments of the disclosure should be interpreted as encompassing all modifications or variations derived based on the technical spirit of various embodiments of the disclosure in addition to the embodiments disclosed herein.

What is claimed is:

1. A display apparatus comprising:
a display;
a user interface;
a memory storing a contrast sensitivity test image and a contour sensitivity test image for measuring a visual acuity level of a user; and
at least one processor configured to:
through the user interface, receive a user input for identifying visual acuity information of the user, in a state in which the contrast sensitivity test image and the contour sensitivity test image are displayed on the display, based on the information stored in the memory,
identify a plurality of sample images having different image qualities and corresponding to a plurality of image types based on the identified visual acuity information, in a state in which the visual acuity information of the user is identified based on the user input,
identify image quality information corresponding to each of the plurality of image types based on a second user input for selecting at least one of the plurality of sample images, in a state in which the plurality of sample images are displayed on the display, and
display an image on the display based on the identified image quality information.

2. The apparatus as claimed in claim 1, wherein the at least one processor is further configured to:
identify first visual acuity information corresponding to a contrast sensitivity of the user based on the user input received through the user interface, in a state in which the contrast sensitivity test image is displayed on the display,
provide, on the display, a contour sensitivity test image corresponding to the first visual acuity information, in a state in which the contour sensitivity test image is identified based on the identified first visual acuity information, and
identify second visual acuity information corresponding to a contour sensitivity of the user based on the user input received through the user interface, in a state in which the contour sensitivity test image corresponding to the first visual acuity information is displayed.

3. The apparatus as claimed in claim 2, wherein the memory is further storing a plurality of contrast sensitivity test images respectively corresponding to a plurality of contrast sensitivity levels, and a plurality of contour sensitivity test images having different contour sensitivities that respectively correspond to the plurality of contrast sensitivity test images, and
wherein the at least one processor is configured to:
identify a contrast sensitivity level corresponding to a first contrast sensitivity test image as the first visual acuity information based on the information stored in the memory, in a state in which a contrast sensitivity of the first contrast sensitivity test image is unidentifiable from the plurality of contrast sensitivity test images based on the user input received through the user interface, and
identify the second visual acuity information based on the user input received through the user interface, in a state in which at least one contour sensitivity test image corresponding to the first visual acuity information is displayed based on the information stored in the memory.

4. The apparatus as claimed in claim 1, wherein the plurality of image types comprise a face type, a building type, a natural scene type, and a graphic type,
wherein the memory is further storing the plurality of sample images having different image qualities and corresponding to the plurality of image types based on the visual acuity information of the user, and
wherein the at least one processor is further configured to identify a plurality of sample images having different image qualities and corresponding to the face type, the building type, the natural scene type, and the graphic type based on the information stored in the memory, in a state in which the visual acuity information of the user is identified based on the user input.

5. The apparatus as claimed in claim 1, further comprising a communication interface,
wherein the memory is further storing a function corresponding to a correlation between the visual acuity information of the user and an image quality of an image, and
wherein the at least one processor is further configured to:
identify the plurality of sample images having different image qualities and corresponding to the plurality of image types, and the image quality information corresponding to each of the plurality of image types, by inputting, into the function, user context information and the identified visual acuity information of the user received through the communication interface, and
identify image quality information corresponding to at least one selected sample image as the image quality information, in a state in which the second user input for selecting at least one of the plurality of sample images is received through the user interface.

6. The apparatus as claimed in claim 5, wherein the at least one processor is further configured to identify image quality information corresponding to one of the at least one selected sample image as the image quality information, in a state in which the user input for selecting one of the plurality of sample images respectively corresponding the plurality of image types is received through the user interface.

7. The apparatus as claimed in claim 1, wherein the memory is further storing a neural network model trained to classify types of the image displayed on the display apparatus, and
wherein the at least one processor is further configured to:
identify a type of the displayed image by inputting information about an image provided on the display into the trained neural network model, and
provide an image having an updated image quality on the display based on the image quality information corresponding to the identified image type.

8. The apparatus as claimed in claim 1, wherein the at least one processor is further configured to provide, on the display, a second user interface for selecting at least one of the plurality of sample images.

9. The apparatus as claimed in claim 8, wherein the at least one processor is further configured to:
provide, on the display, a third user interface including a plurality of sample images corresponding to a first type among the plurality of image types,
identify image quality information corresponding to a first image as image quality information corresponding to the first type, based on a user input received through the user interface for selecting the first image from the plurality of sample images corresponding to the first type,
provide, on the display, a fourth user interface including a plurality of sample images corresponding to a second type among the plurality of image types, and
identify image quality information corresponding to a second image as image quality information corresponding to the second type, based on a user input received through the second user interface for selecting the second image from the plurality of sample images corresponding to the second type.

10. A control method for a display apparatus, the method comprising:
receiving, through a user interface, a user input for identifying visual acuity information of a user, in a state in which a contrast sensitivity test image and a contour sensitivity test image for measuring a visual acuity level of the user are displayed on a display;
identifying a plurality of sample images having different image qualities and corresponding to a plurality of image types based on the identified visual acuity information, in a state in which the visual acuity information of the user is identified based on the user input;
identifying image quality information corresponding to each of the plurality of image types based on a second user input for selecting at least one of the plurality of sample images, in a state in which the plurality of sample images are displayed on the display; and
displaying an image on the display based on the identified image quality information.

11. The method as claimed in claim 10, wherein the receiving comprises:
identifying first visual acuity information corresponding to a contrast sensitivity of the user based on the user input received through the user interface, in a state in which the contrast sensitivity test image is displayed on the display,
providing, on the display, a contour sensitivity test image corresponding to the first visual acuity information, in a state in which the contour sensitivity test image is identified based on the identified first visual acuity information, and
identifying second visual acuity information corresponding to a contour sensitivity of the user based on the user input received through the user interface, in a state in which the contour sensitivity test image corresponding to the first visual acuity information is displayed.

12. The method as claimed in claim 11, further comprising displaying, on the display, a plurality of contrast sensitivity test images respectively corresponding to a plurality of contrast sensitivity levels, and a plurality of contour sensitivity test images having different contour sensitivities that respectively correspond to the plurality of contrast sensitivity test images, based on the information stored in a memory,
wherein, the identifying the first visual acuity information comprises:
identifying a contrast sensitivity level corresponding to a first contrast sensitivity test image as the first visual acuity information based on the information stored in the memory, in a state in which a contrast sensitivity of the first contrast sensitivity test image is unidentifiable from the plurality of contrast sensitivity test images based on the user input received through the user interface, and
wherein the identifying the second visual acuity information comprises:
identifying the second visual acuity information corresponding to the contour sensitivity of the user based on the user input received through the user interface in a state in which at least one contour sensitivity test image corresponding to the first visual acuity information is displayed based on the information stored in the memory.

13. The method as claimed in claim 10, wherein the plurality of image types include a face type, a building type, a natural scene type, and a graphic type, and
wherein the identifying the plurality of sample images having the different image qualities comprises:
identifying the plurality of sample images having different image qualities and corresponding to the face type, the building type, the natural scene type, and the graphic type based on the plurality of sample images having different image qualities that respectively correspond to the plurality of image types mapped to the visual acuity information of the user, stored in a memory, in a state in which the visual acuity information of the user is identified based on the received user input.

14. The method as claimed in claim 10, further comprising identifying the plurality of sample images having different image qualities and corresponding to the plurality of image types, and the image quality information corresponding to each of the plurality of sample images having different image qualities, by inputting, into a function corresponding to a correlation between the visual acuity information of the user and an image quality of an image, user context information and the identified visual acuity information of the user, received through a communication interface,
wherein the identifying the image quality information corresponding to each of the plurality of image types comprises:
identifying image quality information corresponding to at least one selected sample image as the image quality information corresponding, in a state in which the second user input for selecting at least one of the plurality of sample images is received through the user interface.

15. A non-transitory computer-readable recording medium storing computer instructions that cause a display apparatus to perform an operation if executed by a processor of the display apparatus, wherein the operation comprises:
receiving, through a user interface, a user input for identifying visual acuity information of a user, in a state in which a contrast sensitivity test image and a contour sensitivity test image for measuring a visual acuity level of the user are displayed on a display,
identifying a plurality of sample images having different image qualities and corresponding to a plurality of image types based on the identified visual acuity information, in a state in which the visual acuity information of the user is identified based on the user input,
identifying image quality information corresponding to each of the plurality of image types based on a second user input for selecting at least one of the plurality of sample images, in a state in which the plurality of sample images are displayed on the display, and
displaying an image on the display based on the identified image quality information.
